(19) United States
(12) Patent Application Publication
(10) Pub. No.: US 2024/0327363 A1
(43) Pub. Date: Oct. 3, 2024

(54) METHOD OF PREPARING OF ARTHROPODICIDAL OXADIAZINE INTERMEDIATE

(71) Applicant: ADAMA MAKHTESHIM LTD., Beer Sheva (IL)

(72) Inventors: Eran FOGLER, Meitar (IL); Liza PEN, Beer Sheva (IL); Natalia STRIZHIVER, Lenhavim (IL); Chen VAZANA, Ramat-Gan (IL)

(73) Assignee: ADAMA MAKHTESHIM LTD., Beer Sheva (IL)

(21) Appl. No.: 18/578,908

(22) PCT Filed: Jul. 11, 2022
(86) PCT No.: PCT/IL2022/050743

§ 371 Date: Jan. 12, 2024

Related U.S. Application Data
(60) Provisional application No. 63/221,016, filed on Jul. 13, 2021.

(51) Int. Cl. C07D 273/04; C07C 269/06

(52) U.S. Cl. CPC C07D 273/04; C07C 269/06

(57) ABSTRACT

The present invention pertains to a process for preparing of methyl chloroformyl[4-(trifluoromethoxy)phenyl]carbamate of formula I as intermediate in the preparation of arthropodicidal indoxacarb, comprising reaction of compound of formula (IV) with phosgene or phosgene derivative in the presence of phase transfer catalyst, alkaline base and hydrocarbon organic solvent and in the absence of organic base and metal hydride. This invention also pertains to a telescopic process for preparation of methyl chloroformyl[4-(trifluoromethoxy)phenyl]carbamate of formula I comprising preparation of isocyanate of formula (II) by reaction of compound of aniline derivative of formula (III) with phosgene or phosgene derivative in the presence of dimethylcarbonate and hydrocarbon organic solvent; following the preparation of compound of formula (IV) by reaction of compound of formula (II) with methanol and distillation out of dimethylcarbonate and reaction of compound of formula (IV) with phosgene or phosgene derivative in the presence of phase transfer catalyst, alkaline base and hydrocarbon organic solvent.

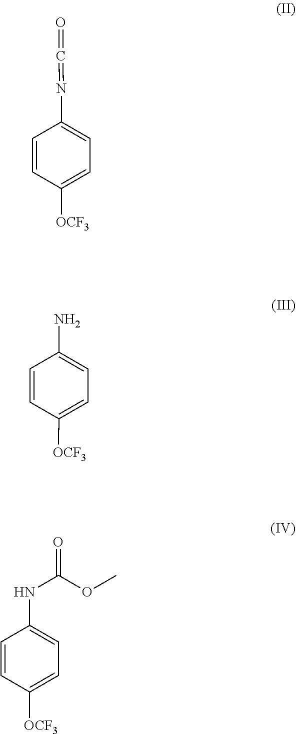 [SMILES: O=C=Nc1ccc(OC(F)(F)F)cc1 ; Nc1ccc(OC(F)(F)F)cc1 ; COC(=O)Nc1ccc(OC(F)(F)F)cc1]

### FIELD OF INVENTION

The present invention concerns a process for preparing of methyl chloroformyl[4-(trifluoromethoxy)phenyl]carbamate as key intermediate for preparation of indoxacarb in the absence of organic base and metal hydride.

### BACKGROUND

Arthropodicidal oxadiazines and the corresponding synthetic methods for the preparation of biologically active oxadiazines and their intermediates are previously disclosed in patent applications WO 9211249, WO 95/29171 and WO 9319045. In particular, the preparation of key intermediate methyl chloroformyl[4-(trifluoromethoxy)phenyl]carbamate using sodium hydride in mineral oil and glyme is disclosed in WO 95/29171. In addition, IN241255 claims the preparation of methyl chloroformyl[4-(trifluoromethoxy)phenyl]carbamate using sodium hydride in aromatic solvent and ether. Furthermore, IN 240984 claims the preparation of methyl chloroformyl[4-(trifluoromethoxy)phenyl]carbamate from the corresponding carbamic acid methyl ester with triphosgene or diphosgene in the presence of organic amine and aromatic solvent.

However, the use of sodium hydride or similar metal hydrides in a large-scale commercial preparations unfavorable for safety reasons. On the other hand, the use of amine bases in large-scale commercial processes have significant impact to the environment and human health. Amine wastes generated from said processes must be treated before they are exposed to the environment. In addition, publication IPCOM000262804D recites the preparation of methyl (4-(trifluoromethoxy)phenyl)carbamate precursor using phosgene, dimethylcarbonate and methanol, however, no further conversion of said precursor to final methyl chloroformyl[4-(trifluoromethoxy)phenyl]carbamate of formula I by telescopic conversion is disclosed.

Based on the above, the aforementioned preparative methods still must be improved for safe economic commercial operation. In particular, the need exists for a more efficient process to prepare methyl chloroformyl[4-(trifluoromethoxy)phenyl]carbamate of formula I as intermediate in the preparation of arthropodicidal indoxacarb.

### SUMMARY

The present invention provides a process for preparation of methyl chloroformyl[4-(trifluoromethoxy)phenyl]carbamate of formula I

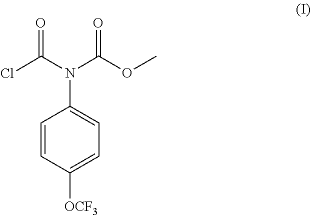 [SMILES: COC(=O)N(C(=O)Cl)c1ccc(OC(F)(F)F)cc1]

comprising reaction of compound of formula (IV) with phosgene or phosgene derivative in the presence of phase transfer catalyst, alkaline base and hydrocarbon organic solvent and in the absence of organic base and metal hydride.

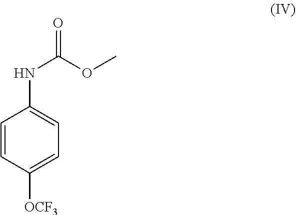 [SMILES: COC(=O)Nc1ccc(OC(F)(F)F)cc1]

In addition, the present invention provides a telescopic process for preparation of methyl chloroformyl[4-(trifluoromethoxy)phenyl]carbamate of formula I

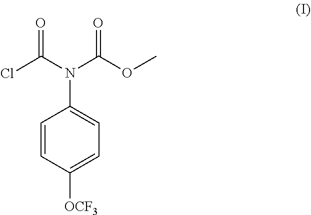 [SMILES: COC(=O)N(C(=O)Cl)c1ccc(OC(F)(F)F)cc1]

comprising:
a) preparation of compound of formula (II)

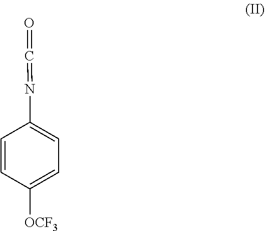 [SMILES: O=C=Nc1ccc(OC(F)(F)F)cc1]

by reaction of compound of formula (III) with phosgene or phosgene derivative in the presence of dimethylcarbonate and hydrocarbon organic solvent

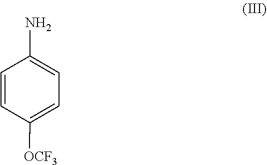 [SMILES: Nc1ccc(OC(F)(F)F)cc1]

b) preparation of compound of formula (IV) by reaction of compound of formula (II) with methanol and distillation out of dimethylcarbonate

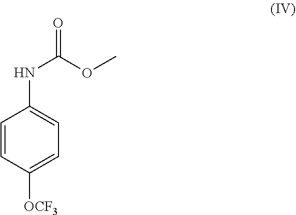 [SMILES: COC(=O)Nc1ccc(OC(F)(F)F)cc1]

c) reaction of compound of formula (IV) with phosgene or phosgene derivative in the presence of phase transfer catalyst, alkaline base and hydrocarbon organic solvent.

The present invention further provides a process of preparation of indoxacarb of formula V

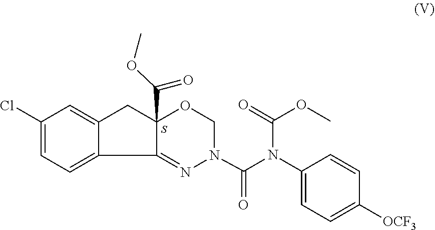

using methyl chloroformyl[4-(trifluoromethoxy)phenyl]carbamate of formula (I) prepared by process, comprising reaction of compound of formula (IV) with phosgene in the presence of phase transfer catalyst, alkaline base and hydrocarbon organic solvent and in the absence of organic base and metal hydride.

### DESCRIPTION

### Definitions

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

The term “a” or “an” as used herein includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one skilled in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages, or proportions, and other numerical values used in the specification and claims, are to be understood as being modified in all instances by the term “about.”

Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In this regard, use of the term “about” herein specifically includes ±10% from the indicated values in the range. In addition, the endpoints of all ranges directed to the same component or property herein are inclusive of the endpoints, are independently combinable, and include all intermediate points and ranges.

Certain compounds of this invention can exist as one or more stereoisomers. The various stereoisomers include enantiomers, diastereomers, and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

Accordingly, the compounds of the invention may be present as a mixture of stereoisomers, individual stereoisomers, or as an optically active form.

The term “telescopic process” as used herein refers to carrying out several reactions without isolating the intermediate products. In particular, telescopic process suggests the execution of multiple transformations (including reaction quenches and other workup operations) without the direct isolation of intermediates. Telescoped solutions of intermediates can be extracted, filtered (as long as the desired product remains in the filtrate), and solvent exchanged, but the intermediate is ultimately held in solution and carried forward to the subsequent transformation.

All publications, patents and patent applications mentioned in this specification are herein incorporated in their entirety by reference into the specification, to the same extent as if each individual publication, patent or patent application was specifically and individually indicated to be incorporated herein by reference.

The present invention provides technology useful for the successful and convenient preparation of methyl chloroformyl[4-(trifluoromethoxy)phenyl]carbamate of formula (I) as intermediate for synthesis of indoxacarb of formula V in the absence of organic base and metal hydride.

According to an embodiment, the present invention concerns the process for preparation of methyl chloroformyl[4-(trifluoromethoxy)phenyl]carbamate of formula I

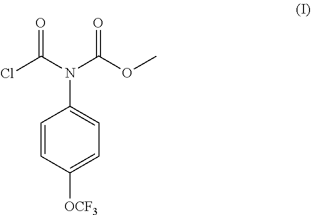 [SMILES: COC(=O)N(C(=O)Cl)c1ccc(OC(F)(F)F)cc1]

comprising reaction of compound of formula (IV) with phosgene or phosgene derivative in the presence of phase transfer catalyst, alkaline base and hydrocarbon organic solvent and in the absence of organic base and metal hydride.

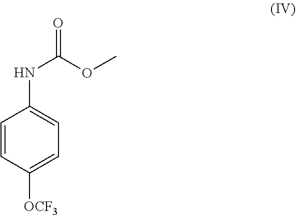 [SMILES: COC(=O)Nc1ccc(OC(F)(F)F)cc1]

In an embodiment phosgene derivative is selected from diphosgene, triphosgene or the mixtures thereof.

In some embodiment, the process carried out “in the absence of organic base” refers to the process including not more than 0.01 mole of organic base on each 1 mole of carbamic acid methyl ester of formula (IV).

In some embodiment, the process carried out “in the absence of organic base” refers to the process including not more than 0.05 mole of organic base on each 1 mole of carbamic acid methyl ester of formula (IV).

In some embodiment, the process carried out “in the absence of organic base” refers to the process including not more than 0.1 mole of organic base on each 1 mole of carbamic acid methyl ester of formula (IV).

In some embodiment, the process carried out “in the absence of metal hydride” refers to the process including not more than 0.01 mole of metal hydride on each 1 mole of carbamic acid methyl ester of formula (IV).

In some embodiment, the process carried out “in the absence of metal hydride” refers to the process including not more than 0.05 mole of metal hydride on each 1 mole of carbamic acid methyl ester of formula (IV).

In some embodiment, the process carried out “in the absence of metal hydride” refers to the process including not more than 0.1 mole of metal hydride on each 1 mole of carbamic acid methyl ester of formula (IV).

According to some embodiment, the process is suitably carried out in hydrocarbon organic solvent such as hexane, petroleum ether, toluene, chlorobenzene, xylene, mesitylene and the mixtures thereof.

In a class of this embodiment the process is carried out in chlorobenzene.

According to some embodiment the process pertains the alkaline base such as sodium hydroxide, potassium hydroxide, potassium carbonate, sodium carbonate and the mixtures thereof.

In a class of this embodiment the process concerns the presence of sodium hydroxide, potassium hydroxide or the mixtures thereof.

According to some embodiment the amount of alkaline base used in the process is 1.0 to 10.0 mole as related to 1 mole of carbamic acid methyl ester of formula (IV).

According to some embodiment the amount of sodium hydroxide base used in the process is 6.0 mole as related to 1 mole of carbamic acid methyl ester of formula (IV).

In some embodiment the process pertains the presence of phase transfer catalyst, such as tetra-butyl ammonium iodide, tetra-ethyl ammonium bromide, tetra-methyl ammonium bromide, tetra-propyl ammonium bromide, tetra-butyl ammonium bromide, crown ethers, PEG and the mixtures thereof.

According to some embodiment the amount of phase transfer catalyst used in the process is 0.001 to 0.1 mole as related to 1 mole of carbamic acid methyl ester of formula (IV).

According to some embodiment the amount of phase transfer catalyst used in the process is 0.05 mole as related to 1 mole of carbamic acid methyl ester of formula (IV).

In a class of this embodiment the phase transfer catalyst is tetra-butyl ammonium bromide (TBAB).

According to another embodiment the compound of formula (II) is not isolated from the reaction mixture.

The desired product, a compound of Formula I, can be isolated by methods known to those skilled in the art, including crystallization, extraction and distillation.

According to an embodiment, the present invention concerns the telescopic process for preparation of methyl chloroformyl[4-(trifluoromethoxy)phenyl]carbamate of formula I

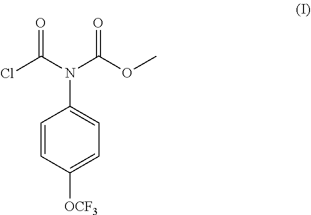 [SMILES: COC(=O)N(C(=O)Cl)c1ccc(OC(F)(F)F)cc1]

comprising:
a) preparation of compound of formula (II)

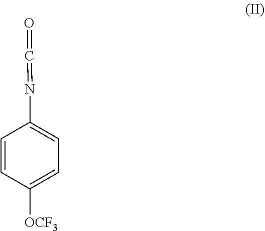 [SMILES: O=C=Nc1ccc(OC(F)(F)F)cc1]

by reaction of compound of formula (III) with phosgene or phosgene derivative in the presence of dimethylcarbonate and hydrocarbon organic solvent

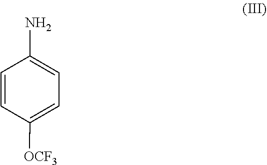 [SMILES: Nc1ccc(OC(F)(F)F)cc1]

b) preparation of compound of formula (IV) by reaction of compound of formula (II) with methanol and distillation out of dimethylcarbonate

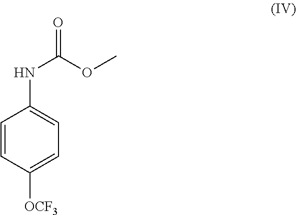 [SMILES: COC(=O)Nc1ccc(OC(F)(F)F)cc1]

c) reaction of compound of formula (IV) with phosgene or phosgene derivative in the presence of phase transfer catalyst, alkaline base and hydrocarbon organic solvent.

In the class of this embodiment the hydrocarbon organic solvent is selected from the group consisting of toluene, chlorobenzene, xylene, mesitylene and the mixtures thereof.

According to some embodiment the preparation of compound of formula (II) is carried out at a temperature of from 10° C. to 70° C.

In a class of this embodiment the preparation of compound of formula (II) is carried out at a temperature of 25 to 70° C.

In a class of this embodiment the preparation of compound of formula (II) is carried out at a temperature of 35° C.

According to some embodiment process comprising from 0.0:1.0 to 0.84:1.0 mole ratio of dimethyl carbonate/chlorobenzene.

In a further embodiment the preparation of compound of formula (III) is carried out at a temperature of from 10° C. to 70° C.

In a further embodiment wherein the preparation of compound of formula (IV) is carried out at a temperature of from 0° C. to 80° C.

In a further embodiment the preparation of compound of formula (I) is carried out at a temperature of from 0° C. to 80° C.

In some another embodiment the present invention concerns the preparation of indoxacarb of formula V

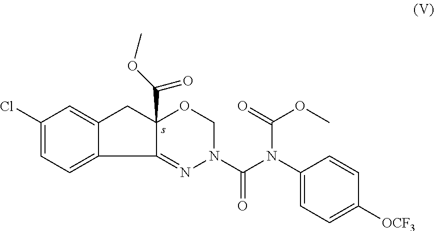 [SMILES: COC(=O)N(C(=O)N1CO[C@@]2(C(=O)OC)Cc3cc(Cl)ccc3C2=N1)c1ccc(OC(F)(F)F)cc1]

using methyl chloroformyl[4-(trifluoromethoxy)phenyl]carbamate of formula I

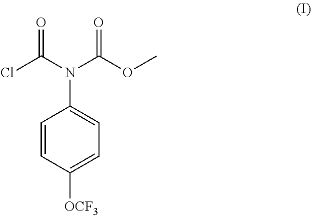 [SMILES: COC(=O)N(C(=O)Cl)c1ccc(OC(F)(F)F)cc1]

prepared by reaction of compound of formula (IV) with phosgene or phosgene derivative in the presence of phase transfer catalyst, alkaline base and hydrocarbon organic solvent and in the absence of organic base and metal hydride as described in previous embodiments.

The desired product, a compound of Formula V, can be isolated by methods known to those skilled in the art, including crystallization, extraction and distillation.

In some another embodiment the present invention concerns the preparation of indoxacarb of formula V

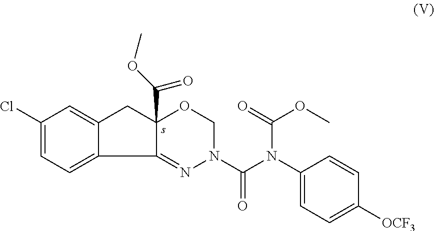 [SMILES: COC(=O)N(C(=O)N1CO[C@@]2(C(=O)OC)Cc3cc(Cl)ccc3C2=N1)c1ccc(OC(F)(F)F)cc1]

using methyl chloroformyl[4-(trifluoromethoxy)phenyl]carbamate of formula I

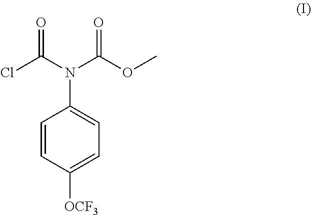 [SMILES: COC(=O)N(C(=O)Cl)c1ccc(OC(F)(F)F)cc1]

prepared by the telescopic process comprising:
a) preparation of compound of formula (II)

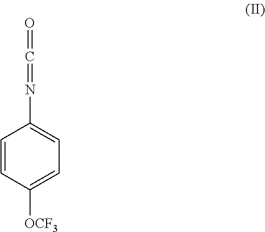 [SMILES: O=C=Nc1ccc(OC(F)(F)F)cc1]

by reaction of compound of formula (III) with phosgene in the presence of dimethylcarbonate and hydrocarbon organic solvent

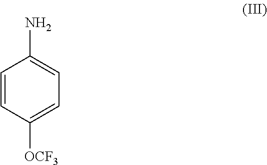 [SMILES: Nc1ccc(OC(F)(F)F)cc1]

b) preparation of compound of formula (IV) by reaction of compound of formula (II) with methanol and distillation out of dimethylcarbonate

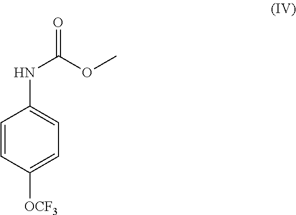 [SMILES: COC(=O)Nc1ccc(OC(F)(F)F)cc1]

c) reaction of compound of formula (IV) with phosgene in the presence or phosgene derivative of phase transfer catalyst, alkaline base and hydrocarbon organic solvent as described in previous embodiments.

The desired product, a compound of Formula I, can be isolated by methods known to those skilled in the art, including crystallization, extraction and distillation.

The present disclosure with reference to certain embodiments described herein, is further illustrated by reference to the following examples describing in detail the preparation of the methyl chloroformyl[4-(trifluoromethoxy)phenyl]carbamate of formula I, compound of formula (II), (IV) and (V). It will be apparent to those skilled in the art that many modifications, both to materials and methods, may be practiced without departing from the scope of the disclosure. Additional embodiments will become apparent to one skilled in the art from consideration of the specification.

### EXAMPLES

### Example 1: Preparation of Methyl chloroformyl[4-(trifluoromethoxy)phenyl]carbamate (Method A)

11 g of chlorobenzene and 0.05 g (0.15 mmol) of TBAB were charged into a three-neck flask with a stirrer. After that 14.3 g (0.09 mol) of 25% aqueous NaOH and solution of 3.5 g (0.015 mol) of carbamic acid methyl ester of formula (IV) with 2.94 g (0.01 mol) of triphosgene in 38.5 g chlorobenzene were added dropwise to the reaction mixture during 1 h at 25° C., then the mixture was stirred for additional 5 minutes. After that 50 ml of 10% aqueous HCl were added and the organic phase was separated and washed with additional 50 ml of 10% aqueous HCl. Then, the organic phase was concentrated under reduced pressure (10 mbar at 40° C.) to afford 4.1 gr of product (yield 86%).

### Example 2: Continuous Flow Preparation of Methyl chloroformyl[4-(trifluoromethoxy)phenyl]carbamate (Method B)

For the continuous flow experiments typically, the experimental set-up involved two syringe pumps followed by a micromixer, which was then connected to a coiled PFA tube (0.8 mm i.d., 3.65 mL volume). The residence time was varied by changing the flow rates. The samples were collected in acidic solution to quench the reaction at the outlet of the reaction tube. The product was subjected to analysis after further dilution.

3.5 g (0.015 mol) of carbamic acid methyl ester of formula (IV), 2.94 g (0.01 mol) of triphosgene and 0.05 g (0.15 mmol) of TBAB were dissolved in 50 g chlorobenzene and 3.6 g of NaOH was dissolve in 11 ml of water. The two reacting solutions were mixed using T micro mixer followed by retention time tube. The process was carried out at 25° C. The residence time in the tube was maintained at 5 minutes. After that the reaction mixture was washed with 10% aqueous HCl, the organic phase was separated and concentrated under reduced pressure (10 mbar at 40° C.) to afford 0.5 gr of product (yield 98%)

### Example 3: Continuous Flow Preparation of Methyl chloroformyl[4-(trifluoromethoxy)phenyl]carbamate (Method B)

For the continuous flow experiments typically, the experimental set-up involved two syringe pumps followed by a micromixer, which was then connected to a coiled PFA tube (0.8 mm i.d., 3.65 mL volume). The residence time was varied by changing the flow rates. The samples were collected in acidic solution to quench the reaction at the outlet of the reaction tube. The product was subjected to analysis after further dilution.

3.5 g (0.015 mol) of carbamic acid methyl ester of formula (IV), 10.9 g (0.01 mol) of 27% phosgene solution in MCB and 0.05 g (0.15 mmol) of TBAB were dissolved in 50 g chlorobenzene and 3.6 g of NaOH was dissolve in 11 ml of water. The two reacting solutions were mixed using T micro mixer followed by retention time tube. The process was carried out at 25° C. The residence time in the tube was maintained at 5 minutes. After that the reaction mixture was washed with 10% aqueous HCl, the organic phase was separated and concentrated under reduced pressure (10 mbar at 40° C.) to afford 0.38 gr of product (yield 74%)

1. A process for preparation of methyl chloroformyl[4-(trifluoromethoxy)phenyl]carbamate of formula I 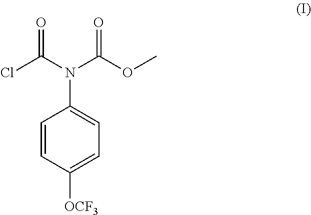 [SMILES: COC(=O)N(C(=O)Cl)c1ccc(OC(F)(F)F)cc1]
comprising reaction of compound of formula (IV) with phosgene, di phosgene or triphosgene in the presence of phase transfer catalyst, alkaline base and hydrocarbon organic solvent and in the absence of organic base and metal hydride.

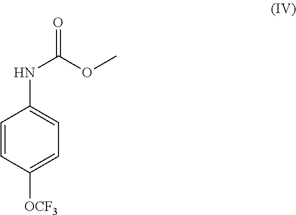 [SMILES: COC(=O)Nc1ccc(OC(F)(F)F)cc1]

2. The process according to claim 1 wherein the hydrocarbon organic solvent is selected from the group consisting of hexane, petroleum ether, toluene, chlorobenzene, xylene, mesitylene and the mixtures thereof.

3. The process according to claim 1 wherein the alkaline base is selected from the group consisting of sodium hydroxide, potassium hydroxide, potassium carbonate, sodium carbonate and the mixtures thereof.

4. The process according to claim 1 wherein phase transfer catalyst is selected from the group consisting of tetra-butyl ammonium iodide, tetra-ethyl ammonium bromide, tetra-methyl ammonium bromide, tetra-propyl ammonium bromide, tetra-butyl ammonium bromide, crown ethers, PEG and the mixtures thereof.

5. The process according to claim 4, wherein the phase transfer catalyst is tetra-butyl ammonium bromide (TBAB).

6. The process according to claim 1 wherein the compound of formula (IV) is not isolated from the reaction mixture.

7. A telescopic process for preparation of methyl chloroformyl[4-(trifluoromethoxy)phenyl]carbamate of formula I 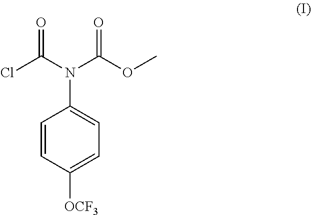 [SMILES: COC(=O)N(C(=O)Cl)c1ccc(OC(F)(F)F)cc1]
comprising:
a) preparation of compound of formula (II) 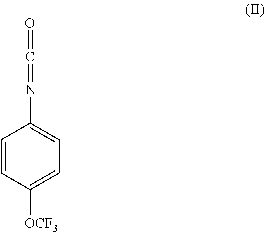 [SMILES: O=C=Nc1ccc(OC(F)(F)F)cc1]
by reaction of compound of formula (III) with phosgene in the presence of dimethylcarbonate and hydrocarbon organic solvent 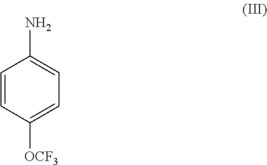 [SMILES: Nc1ccc(OC(F)(F)F)cc1]
b) preparation of compound of formula (IV) by reaction of compound of formula (II) with methanol and distillation out of dimethylcarbonate 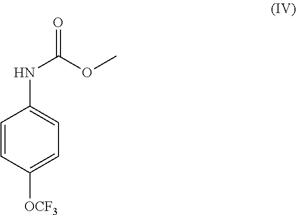 [SMILES: COC(=O)Nc1ccc(OC(F)(F)F)cc1]
c) reaction of compound of formula (IV) with phosgene in the presence of phase transfer catalyst, alkaline base and hydrocarbon organic solvent.

8. The process according to claim 7 wherein hydrocarbon organic solvent is selected from the group consisting of toluene, chlorobenzene, xylene, mesitylene and the mixtures thereof.

9. The process according to claim 7, wherein the preparation of compound of formula (II) is carried out at a temperature of from 10° C. to 70° C.

10. The process according to claim 8, comprising from 0.0:1.0 to 0.84:1.0 of dimethyl carbonate/chlorobenzene.

11. The process according to claim 7, wherein the preparation of compound of formula (III) is carried out at a temperature of from 10° C. to 70° C.

12. The process according to claim 7, wherein the preparation of compound of formula (IV) is carried out at a temperature of from 0° C. to 80° C.

13. The process according to claim 1, wherein the preparation of compound of formula (I) is carried out at a temperature of from 0° C. to 80° C.

14. The process of preparation of indoxacarb of formula V 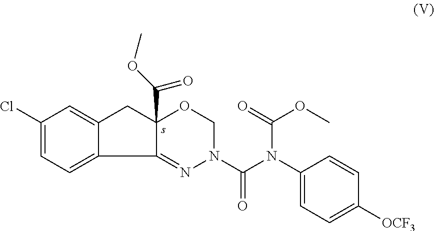 [SMILES: COC(=O)N(C(=O)N1CO[C@@]2(C(=O)OC)Cc3cc(Cl)ccc3C2=N1)c1ccc(OC(F)(F)F)cc1]
using compound of formula (I) prepared by claims 1-7.
